(19) United States
(12) Patent Application Publication
(10) Pub. No.: US 2007/0258956 A1
(43) Pub. Date: Nov. 8, 2007

(54) Methods and apparatus for promoting hair growth using adipose cell based therapies

(75) Inventors: Joel C. Higgins, Claypool, IN (US); Felipe Palacios, Pearland, TX (US); Nicolaas Vermeulen, North Manchester, IN (US)

Correspondence Address: HARNESS, DICKEY & PIERCE, P.L.C., P.O. BOX 828, BLOOMFIELD HILLS, MI, 48303, US

(73) Assignee: Biomet Manufacturing Corp., Warsaw, IN (US)

(21) Appl. No.: 11/417,302

(22) Filed: May 2, 2006

(51) Int. Cl. A61K 35/12; C12N 5/08

(52) U.S. Cl. 424/93.7; 435/371

(57) ABSTRACT

Methods and compositions for use in the treatment of androgenetic alopecia and other forms of hair loss by inducing and expediting the growth of hair in the scalp of a patient. Methods comprise the steps of isolating adipose-derived cells and hair follicles from the patient and implanting the combined cells and follicles into the scalp in areas requiring new growth of hair. Use of differentiated and undifferentiated adipose-derived cells with processed and grafted hair follicles for the therapeutic and cosmetic treatment of hair loss in vivo is disclosed.

### BACKGROUND

The present invention relates to methods and apparatus for inducing and expediting hair growth. In particular, methods include those using adipose-derived stem cells.

Recent studies suggest that hair follicles are in a constant flux between an active growth stage, an involution stage and a resting stage. It has been demonstrated that this follicle “cycling” is heavily dependent on the activity of a subset of follicle stem cells. Follicle stem cells are located in the top one third of a mature hair follicle during anagen or the follicle's growth phase and have been characterized generally as epithelial stem cells. Follicle stem cells have the capacity to respond to signals originating from dermal cells to induce the growth of new hair follicle cells, thus promoting hair self-renewal. More importantly, hair follicle cycling and therefore, hair growth, is highly dependent on the activities of these hair follicle stem cells.

Hair loss including androgenetic alopecia, also known as male-pattern or female-pattern hair loss, represents approximately fifty percent of cases involving hair loss in the United States. Hair loss may also be caused by environmental and genetic factors unrelated to androgens including alopecia greata, permanent alopecia, anagen effluvium, lichen planopilaris and discoid lupus erythematosus.

Current methods for treating hair loss include pharmaceutical drugs to inhibit the effects of androgens. Individuals who are genetically susceptible to the effects of androgens experience shortened hair growth cycles, which manifest in thinning and bitemporal recession of hair. Importantly, humans are born with a finite number of approximately 100,000 terminal hair follicles on the scalp. Pharmaceutical treatments used in androgenetic alopecia stimulate existing hair follicles to produce thicker hair and prevent hair thinning.

There are two pharmaceuticals approved by the Food and Drug Administration for the treatment of androgenetic alopecia: topical minoxidil (Rogaine®, Pfizer) and oral Finasteride (Propecia®, Merck). Both drugs attack the effects of dihydrotestosterone. However, neither drug restores all the hair, no new follicles are produced, treatment requires prolonged usage of the drug, and if treatment is stopped any benefits gained will be lost and the hair thickness will regress to levels as if no treatment was undertaken. Reversible side effects associated with the use of these androgen inhibitors have been reported throughout the scientific literature including: decreased libido, erectile dysfunction and dermatologic discomfort. Other modalities for treating hair loss include in vivo hair transplantation whereby donor hair follicles from areas of the scalp that are insensitive to the effects of androgens are transplanted to areas experiencing irregular hair follicle cycling or areas with few hair follicles.

### SUMMARY OF THE INVENTION

The present invention provides methods for inducing and expediting hair growth by implanting a combination of adipose-derived multipotent cells with hair follicles that have the potential to expedite the growth of implanted follicles and create new follicles where none previously existed. Methods include:

(a) isolating adipose-derived stem cells from adipose tissue;

(b) combining said stem cells with processed hair follicles; and

(c) implanting the combined stem cells and processed hair follicles into the skin. In various embodiments, the stem cells are implanted with an angiogenic factor such as platelet rich plasma.

In various embodiments, the adipose tissue is harvested by liposuction, and the lipoaspirate is further treated to yield adipose-derived stem cells. The stem cells may then be further combined with hair follicles and a biocompatible carrier. In various embodiments, the stem cells are implanted with hair and hair follicles in hair transplantation techniques including those known in the art. Methods are also provided for interoperative harvest and implantation of stem cells and autologous hair follicles to the site of acute and chronic hair loss caused by androgenetic alopecia, alopecia areata and hair loss due to chemotherapy and radiation therapy.

### DESCRIPTION

The headings (such as “Introduction” and “Summary”) used herein are intended only for general organization of topics within the disclosure of the invention, and are not intended to limit the disclosure of the invention or any aspect thereof. In particular, subject matter disclosed in the “Introduction” may include aspects of technology within the scope of the invention, and may not constitute a recitation of prior art. Subject matter disclosed in the “Summary” is not an exhaustive or complete disclosure of the entire scope of the invention or any embodiments thereof.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations of the stated features. Specific examples are provided for illustrative purposes of how to make, use and practice the compositions and methods of this invention and, unless explicitly stated otherwise, are not intended to be a representation that given embodiments of this invention have, or have not, been made or tested.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the word “include,” and its variants, is intended to be non-limiting, such that recitation of items in a list is not to the exclusion of other like items that may also be useful in the materials, compositions, devices, and methods of this invention.

ADSSCs and Stem Cells

The present invention provides composition and methods for hair growth using adipose-derived stem cells including adipose-derived stromal and stem cells or “ADSSCs.” “ADSSCs” refers to any pluripotent/multipotent cell that originates from adipose tissue, preferably comprising stem cells. By “adipose,” is meant any fat tissue, either white or brown adipose tissue-derived from subcutaneous, omental/visceral, mammary, gonadal, or other adipose tissue site.

In various embodiments, adipose cells are derived from human subcutaneous fat isolated by suction assisted lipectomy or liposuction. The stem cells may be isolated using any suitable method, including methods known in the art such as mechanical and breakdown centrifugation. In one embodiment, stem cells are isolated using enzymatic digestion. In one embodiment, ADSSCs are isolated from lipoaspirate, treated by sonication, and enriched by centrifugation. The ADSSCs are preferably isolated from the liquid material and are subsequently washed and pelleted.

In one embodiment, isolation comprises collecting about 50 cc of adipose tissue by suction-assisted tumescent liposuction inside a specialized collection container attached to suction hoses and to a liposuction cannula. The collection container has a gauze-type grid filter that allows the tumescent fluid to pass through and retains the solid adipose tissue containing the stromal and stem cell population. After collecting the adipose tissue, the collection container is removed from the suction device and reattached to a centrifugation device. The filter unit further contains a 100 micrometer pore size filter. Once the collection container containing the adipose tissue is attached to the centrifugation device, the tissue is sonicated. After sonication, the entire apparatus is inserted into a centrifuge bucket and centrifuged at, for example, 300×g for 5 minutes. After centrifugation, the collection container together with the filter unit is detached and can be discarded. The pellet containing the adipose stromal and stem cells can then be resuspended in biocompatible solutions such as autologous plasma, plasma concentrate and platelet rich plasma, combined with processed hair follicles, combined with excised hair follicles for implantation, or resuspended in culture medium for propagation and differentiation.

Prior to implantation, ADSSCs can be undifferentiated or differentiated. In various embodiments, the ADSSCs can be differentiated into at least one of: epithelial stem cells, matrix cells, clonogenic keratinocytes, dermal cells or combinations thereof. The invention provides methods for differentiating isolated ADSSCs into follicle stem and dermal cells comprising culturing ADSSCs in a culture medium which can comprise a composition capable of supporting the growth and differentiation of ADSSCs into cells capable of regenerating new hair follicles. In various embodiments, isolated ADSSCs are stably or transiently transfected or transduced with nucleic acid of interest using a plasmid, naked DNA, viral vectors (retro viruses, adenoviruses, adeno-associated viruses or other viral vectors) or alternate vector embodiments. Nucleic acids of interest include, but are not limited to, those which encode gene products that stimulate the production of epithelial and dermal cell growth signals present in hair follicles, particularly, follicle neogenesis signals and signals expressed by follicle stem cells and dermal cells at the onset of hair follicle growth (anagen). Examples include, but are not limited to: integrins, epithelial stem cell markers (including: CD 34, S100a4 and S100a6), growth factors and their cognate receptors including: TGF (transforming growth factor), IGF (insulin-like growth factor), PDGF (platelet-derived growth factor), EGF (epidermal growth factor), AFBF (acidic fibroblast growth factor), bFBF (basic fibroblast growth factor), HGF (hepatocytic growth factor), KGF (keratocyte growth factor), VEGF (Vascular endothelial growth factor), Neuropeptides, Proteoglycans (including versican) and other regulatory molecules including BMPs, Noggin, Wnt, Hedgehog, TNF family and catenins, and combinations thereof. However, these particular growth factors are not limiting. Any polypeptide growth factor capable of inducing hair follicle neogenesis or which would allow the ADSSCs to differentiate into regenerative hair follicle stem cells, hair follicle epithelial cells and dermal cells and support the growth and development of implanted hair follicles may be useful in the practice of the invention. In some embodiments the growth factors can be derived from any physiologically active source, including, but not limited to autologous, allogeneic or xenogeneic sources.

Follicle stem cells have been shown to reconstitute the epidermal layer, the sebaceous gland and the hair follicle itself. It is contemplated that undifferentiated and differentiated ADSSCs can provide the stimulatory signals required for epithelial-mesenchymal interactions that occur prior to the onset of new hair growth when placed in close proximity with hair follicle dermal cells. Alternatively, it is contemplated that when stimulated by the appropriate morphogenic signals from the patient's injected or transplanted follicles, ADSSCs can respond by differentiating into hair follicle cells capable of regenerating new follicles, or secrete bioactive factors that are conducive to the formation of new hair follicles. In various embodiments, the angiogeneic effect required for development of the newly transplanted hair follicles and/or ADSSCs can be provided by implanting platelet rich plasma and/or platelet concentrate together with the hair follicles and ADSSCs. In various embodiments, platelet rich plasma is a source of endogenous morphogenic growth factors that can be used to stimulate the development of new blood vessels required for the growth of new and existing hair follicles, comprising growth factors such as vascular endothelial growth factor (VEGF) contained within the platelet's alpha-granules. Several devices exist in the art that are capable of producing a platelet rich plasma sample that can be used and implanted intraoperatively in methods of this invention.

In various embodiments, the processed hair follicles are a source of hair follicle cells that are capable of expediting hair growth and support the growth of new hair follicles when combined with ADSSCs isolated from the host subject. Hair follicle stem cells derived from the bulge region of the hair follicle are thought to consist of a subgroup of stem cells having characteristic properties of epithelial stem cells.

Methods

The present invention provides methods for inducing and expediting hair growth using a combination of ADSSCs, hair follicles and, optionally, platelet growth factors. Methods for the growth of hair and expediting hair growth include cosmetic and therapeutic procedures. Therapeutic procedures include those for growth of hair following conditions that result in hair loss and baldness as a result of inflammation, autoimmune disease, radiation and chemotherapies, burns and other trauma.

The interoperative methods disclosed here are applicable to any human or other animal species. In various embodiments, the methods comprise the induction and promotion of human hair growth. In other embodiments, the methods comprise the induction and promotion of non-human hair growth.

In various embodiments, methods of this invention include the steps of isolating a regenerative cell rich fraction from lipoaspirate material, preferably enriched in ADSSCs, followed by combining the ADSSCs with isolated hair follicles. In various embodiments, the hair follicles are processed. Optionally, the ADSSCs are also combined with platelet concentrate or platelet rich plasma. The ADSSCs are preferably combined with processed hair follicles from the donor and injected into the skin (e.g., scalp) of the donor in areas requiring hair growth. In various embodiments, ADSCCs can be implanted with androgen insensitive hair follicles to induce proliferation of hair follicles and accelerate the growth of implanted hair follicles. In some embodiments, methods further comprise treating the ADSSCs with bioactive agents that promote and enhance proliferation, differentiation and commitment of ADSSCs into hair follicle cells or their supporting dermal components, either in vitro or in vivo, as discussed above.

Processed hair follicles may be obtained through a variety of methods, including those known in the art. In various embodiments, methods are carried out by incorporating the ADSSCs into established hair transplanting methodologies. In one embodiment, the isolated ADSSCs are combined with excised hair follicles that are associated with hair grafts and implanted according to established hair transplantation techniques. Hair transplantation procedures are well known in the art. See for example: Epstein, J. S (2003) “Hair Transplantation for Men with Advanced Degree of Hair Loss.” Plastic & Reconstructive Surgery, 111(1): 414-421, the disclosure of which is incorporated by reference herein. Excised hair follicle grafts are isolated and mixed with high-density cell samples of ADSSCs prior to transplantation.

In various embodiments, such methods include those performed by experts in the field of hair transplantation. In some embodiments, the hair transplantation can involve removing small portions of hair-bearing scalp grafts from a donor and relocating the grafts to a patient in need of hair replacement, including bald and/or thinning areas. The grafts can be of any shape, including round grafts containing from about 5 to 20 hairs and hair follicles. In various embodiments, mini-grafts may contain from about 2 to about 5 hairs and hair follicles, and micro-grafts may contain one or two hairs and hair follicles. In various embodiments, the transplanted graft can be described as a slit graft, which is a transplanted graft inserted into slits created by the medical or hair practitioner containing from about 3 to about 12 hairs and hair follicles. In various embodiments, strip grafts can be used to transplant hair grafts that are long and thin, containing from about 25 to about 45 hairs and hair follicles.

In various embodiments, the grafts can be implanted about one-eighth of an inch apart on the scalp of the recipient. In subsequent transplantation procedures, the patient can have the spaces between the plugs filled with additional grafts. Healthy circulation in the scalp can be maintained by implanting the hairs and hair follicles and ADSSCs with a platelet rich plasma, and/or platelet concentrate to encourage the development of blood vessels in and around the newly transplanted hair grafts.

Compositions

In various embodiments, the present invention provides compositions for hair growth in a human or other animal subject, comprising:

(a) adipose-derived stromal cells and stem cells;

(b) hair follicles; and

(c) a biocompatible carrier optionally comprising autologous platelet rich plasma; wherein the ADSSCs are derived from the patient's adipose tissue. As referred to herein, a “biocompatible carrier” is a material that contains or supports ADSSCs, preferably enabling their growth and inductive properties at the site of injection and/or transplantation which is suitable for use with humans and/or animals without adverse side effects (such as toxicity, irritation, and allergic response) commensurate with a reasonable risk/benefit ratio. The nature of the carrier will depend on the implantation procedure. In one embodiment, the ADSSCs and the processed hair follicles are mixed with the carrier prior to injection into the scalp. In other embodiments the biocompatible carrier may comprise of materials that are compatible with transplantation techniques commonly known in the art.

Biocompatible carriers include those selected from the group comprising calcium alginate, agarose, fibrin, collagen, gelatin, fibronectin, polyglycolic acid, polylactic acid, hyaluronic acid, polyethylene glycol, chondroital, dermatan, polysaccharides, mucopolysacharides, hydrogels, dextrin, amylose, proteins, glycoproteins and any copolymer and other derivatives thereof, platelet rich plasma, platelet concentrate and mixtures thereof. In various embodiments, suitable carrier materials include platelet rich plasma or plasma concentrate.

The present invention is further illustrated through the following non-limiting example.

### EXAMPLE 1

After the isolation of ADSSCs from a human subject, the cells are washed in physiological phosphate buffered saline and placed in biocompatible solutions that are capable of ensuring the integrity of the cells which are commonly known to those of ordinary skill in the art. The adipose-derived stromal and stem cells' suspension is then mixed with processed hair follicles that have been excised from the subject of the adipose-derived stromal and stem cells. The cells and the hair follicles are then resuspended and diluted to any appropriate stromal/stem cell/hair follicle ratio. The mixture of cells and processed hair follicles is then injected into the scalp of the subject after local anesthesia has been administered. The mixture of hair follicles and ADSSCs is injected at each site in a volume of about 200 to about 5 microliters, more preferably from about 100 to about 50 microliters, most preferably, from about 5 to about 25 microliters.

The examples and other embodiments described herein are exemplary and not intended to be limiting in describing the full scope of compositions and methods of this invention. Equivalent changes, modifications and variations of specific embodiments, materials, compositions and methods may be made with substantially similar results.

1. A method for promoting hair growth in a human or other animal subject comprising:
a. combining adipose-derived stromal and stem cells with autologous hair follicles; and
b. implanting the combined adipose-derived cells and autologous hair follicles into skin of said subject.

2. (canceled)

3. A method according to claim 1, wherein the adipose-derived stromal and stem cells are isolated from lipoaspirate.

4. A method according to claim 1, wherein said autologous hair follicles are processed hair follicles.

5. A method according to claim 4, wherein said processed hair follicles comprise at least one of bulge cells, dermal sheath cells, matrix cells, dermal papilla cells, keratinocytes, and epithelial stem cells.

6. A method according to claim 1, wherein said combined adipose-derived stromal and stem cells and autologous hair follicles are combined with an angiogenic factor.

7. A method according to claim 6, wherein said angiogenic factor is platelet rich plasma or platelet concentrate.

8. A method for inducing and expediting hair growth in a human or other animal subject suffering from alopecia comprising:
a. isolating adipose-derived stromal and stem cells obtained from the subject by liposuction from adipose tissue;
b. combining the isolated adipose-derived stromal and stem cells with processed autologous hair follicles; and
c. implanting the combined cells and processed autologous hair follicles to the scalp of said subject.

9. A method according to claim 8, wherein implanting step (c) is performed atraumatically.

10. A method according to claim 8 wherein implanting step (c) is performed by injection.

11. A method according to claim 8, wherein said adipose-derived stromal and stem cells are undifferentiated.

12. A method according to claim 8, wherein adipose-derived stromal cells and stem cells are differentiated prior to said implanting.

13. A method according to claim 12, wherein said adipose-derived stromal cells and stem cells are differentiated by incubation of the cells with a bioactive inducing agent selected from the group consisting of nucleic acids and proteins encoding growth factors, growth factor receptors, transcription factors and related genes, cell surface proteins, structural proteins, ion channel related proteins, morphogenic signaling proteins, and combinations thereof.

14. A method according to claim 13, wherein said bioactive inducing agent is selected from the group consisting of TGF-β1 (transforming growth factor-beta-1), TGF-β2 (transforming growth factor-beta-2), TGF-β3 (transforming growth factor beta-3), IGF (insulin-like growth factor), PDGF (platelet-derived growth factor), EGF (epidermal growth factor), AFBF (acidic fibroblast growth factor), bFBF (basic fibroblast growth factor), HGF (hepatocytic growth factor), KGF (keratocyte growth factor), inhibin A, keratin (KRT2-6), and mixtures thereof.

15. A method according to claim 8, wherein adipose-derived stromal and stem cells are autologous with said subject.

16. A method according to claim 8, wherein adipose-derived stromal and stem cells are heterologous or allogeneic.

17. A method according to claim 8, wherein said combined adipose-derived stromal and stem cells and hair follicles are combined with an angiogenic factor.

18. A composition for inducing and expediting hair growth in a human or other animal subject comprising:
a. adipose-derived stromal and stem cells;
b. hair follicles selected from processed hair follicles excised hair transplant hairs, follicular unit grafts, and combinations thereof; and
c. a biocompatible carrier.

19. A composition according to claim 18, wherein said adipose-derived stromal cells and stem cells are isolated by liposuction of adipose tissue, subjecting the adipose tissue to sonic or electromagnetic energy, and centrifuging the sonicated tissue to form a pellet comprising stromal cells.

20. A composition according to claim 19, wherein the biocompatible carrier comprises autologous platelet rich plasma, platelet concentrate, or combinations thereof.

21. An interoperative method for treating a human or other animal subject having chronic or acute alopecia comprising:
removing fat tissue from the subject;
isolating adipose-derived stromal and stem cells from said tissue;
combining said isolated adipose-derived stromal and stem cells with autologous hair follicles and a biocompatible carrier; and
implanting the adipose-derived stromal and stem cells, autologous hair follicles and biocompatible carrier to the site of hair loss.

22. A method according to claim 21, wherein said adipose-derived stromal and stem cells are differentiated prior to said implanting.

23. A method according to claim 21, wherein the biocompatible carrier comprises autologous platelet rich plasma or platelet concentrate or combinations thereof.
